(19) United States
(12) Patent Application Publication
(10) Pub. No.: US 2023/0323482 A1
(43) Pub. Date: Oct. 12, 2023

(54) APPARATUS, DEVICE AND METHOD FOR PHOTODETECTION OF PATHOGENS

(71) Applicant: RESOLVE DIAGNOSTICS PVT. LTD., Mumbai (IN)

(72) Inventors: Ritwik DAHAKE, Bengaluru (IN); Monika BHATKHANDE, Mumbai (IN); Ram KHANNA, Mumbai (IN)

(73) Assignee: RESOLVE DIAGNOSTICS PVT. LTD., Mumbai (IN)

(21) Appl. No.: 18/042,849

(22) PCT Filed: Aug. 21, 2021
(86) PCT No.: PCT/IN2021/050805

§ 371 Date: Feb. 24, 2023

(30) Foreign Application Priority Data
Aug. 24, 2020 (IN) 202021036287

(51) Int. Cl. C12Q 1/70; B01L 7/00

(52) U.S. Cl. CPC C12Q 1/70; B01L 7/52; B01L 2200/16; B01L 2300/0832; B01L 2300/027; B01L 2300/023; B01L 2300/1894; B01L 2300/0663; B01L 2300/024

(57) ABSTRACT

Described herein is novel, economical device for photodetection of presence of a target nucleic acid in a sample. Accordingly, the detection takes place based on difference in light intensities. The invention further describes the method and apparatus for detection of target nucleic acid using said device.

### FIELD OF INVENTION

The present invention is in field of healthcare. More particularly, the present invention relates to a portable apparatus and a device for economic and rapid collection and photodetection of pathogens in a biological sample and a method thereof.

### BACKGROUND OF INVENTION

India faces the challenge of a range of infectious diseases. The country has witnessed outbreaks and epidemics of many infectious diseases, thereby creating huge pressure on under developed healthcare system. While deaths from non-communicable diseases (NCDs) are becoming increasingly prevalent (NCDs accounted for 5.2 million deaths, 61.8 percent of all deaths in India in 2016), a large number of Indians still continue to die annually due to infectious diseases. This has put a substantial amount of stress on an underfunded and under-resourced health system.

There have been major epidemic diseases such as plague, leprosy, cholera and malaria in the past and despite the challenges faced, significant successes against them have also been achieved. This success also can soon turn into failure if infectious diseases are allowed to go unchecked and begin to show resurgence and thus it will diminish any progress India has made towards its elimination targets.

Among the Indian states, infectious and associated diseases account for around 14% to 43% of total disease burden of the country. Many are treated incorrectly due to inaccurate or delayed diagnostic results. Errors by diagnostic labs end up being a costly affair to the health of the patients considering the risks it poses to them. False reports further complicate the situation and result in delays of the treatment or completely wrong treatment administered by the doctor. Apart from this, analytical errors in diagnostic reports can be a monetary burden on the patients as well.

Although India has progressed immensely in the medical facilities & healthcare system, the use of Point of Care (POC) diagnostic devices is still in its early phase. POC diagnostic equipment is used to obtain diagnostic results from the patient or near the patient. These devices give quick feedback on many sorts of medical tests. POC diagnostic devices are specifically used in five different healthcare settings such as in homes, communities, clinics, peripheral laboratories, and hospitals. POC diagnostic equipment is currently used to determine glucose and cholesterol levels, electrolyte and enzyme tests, drug abuse, infectious disease test, and pregnancy testing. Blood, gas, heart markers, and faecal occult blood tests can also be performed using POC diagnostic equipment. One of the key advantages of doing the tests at the point of care is the speed at which these tests can be performed, quick results and the faster implementation of therapy if needed, based on the results delivered. These diagnostic devices are portable and convenient to carry around even to remote places with underdeveloped infrastructure. They are fairly accurate and are able to achieve real-time, lab-quality diagnostic results within minutes rather than hours using the traditional high end clinical diagnostics machines.

A major drawback of POC test is its sensitivity, which is uncertain for few of the POC tests. Despite this drawback, the rising demand for rapid diagnostics in the healthcare sector has led to the research and development of numerous POC diagnostic equipment and technologies. Researchers around the world are studying and reporting new technologies that can be used in POC diagnostic equipment. In addition to detecting biomarkers of heart failure and blood sugar, there is a great need for a POC method for detecting infectious diseases.

In December 2019, the World Health Organisation (WHO) has declared public health emergency concerned with an outbreak of novel coronavirus (2019-nCoV) i.e., severe acute respiratory syndrome coronavirus-2 (SARS-CoV-2). COVID-19 has become a global pandemic affecting huge population worldwide resulting in financial, economic, social and mental stress. Early detection and prompt treatment are required to not only break the chain of this highly infectious disease but also crucial to provide the best health care to a patient and to reduce the risk of further spreading. Not only COVID-19, this is applicable to other infectious diseases also such as malaria, tuberculosis (TB) and human immunodeficiency virus (HIV). For this reason, many different and new techniques have been researched and developed under the separate category, Rapid Diagnostics Tests (RDT).

The biggest advantage of RDT is that they offer low manufacturing cost and provide the first-level or mass screening to determine the necessity of follow-up tests. But they come at a trade-off. The RDT method lacks complete accuracy and quantification to obtain low-cost and ease of use. Antigen-based detection devices usually have non-specific associations, leading to false alarms and poor accuracy (more than 50% false negatives). In addition, most antigen-based devices require relatively high levels of pathogens in the patient sample compared to Polymerase Chain Reaction (PCR) for detection of a positive result. The WHO recommends at least a 75% panel detection score, the detection success rate for samples with low parasite density (200 parasites/μl) and false positive results less than 10%.

In addition, some RDTs are not quantifiable and can only give positive or negative results. Some RDT also fail to report the infection because the antigen and antibody biomarkers are not present in that phase of disease. The only diagnosis mechanism that can measure the presence of infection in that phase is nucleic acid-based.

According to the Centre for Disease Control and Prevention (CDC) and WHO, for diseases such as Covid-19, Malaria and HIV, the use of PCR is highly recommended to obtain results and to determine the proper treatment, because PCR reveals both the presence and the quantity of the pathogen and more importantly, they have high sensitivity and specificity. However, because these methods are not easier to use, costly and are highly inaccessible at remote places, alternative methods (such as RDT) are often used for diagnosis.

Nucleic acid detection and immunoassay methods are currently one of the most popular methods for detecting infections directly from the source. Nucleic acid detection is usually highly sensitive, but it can be time-consuming, expensive, and requires trained personnel. The use of an isothermal amplification system for detection can help shorten the response time of the results and hardware cost, which makes it attractive for point-of-use applications or when high-volume/responsive testing is required. Alternatively, immunoassays provide reliability and reduce costs. Additionally, some immunoassay formats, such as that using lateral-flow technology, can generate results very quickly. However, immunoassays generally cannot provide sensitivity comparable to nucleic acid-based detection methods.

A key element of nucleic acid-based detection is polymerase chain reaction (PCR) which utilises multi-stage temperature changes and polymerase used to amplify DNA strands (Mulliset al., 1986). The standard polymerase used in PCR can only synthesise from a DNA template, while the RNA amplification requires the use of an enzyme with reverse transcription activity (Bustin, 2000). PCR and reverse transcription PCR are widely used methods for detecting DNA and RNA viruses, respectively (Metcalf, 1995; Pring-Åkerblom et al., 1997; Burkhalter and Savage, 2017; Wadhwa et al., 2017; Liu et al., 2018; Lin et al., 2020).

The quantitative PCR (qPCR) is a robust technique that amplifies the target DNA sequence, while monitoring the signal amplitude from an oligonucleotide probe, usually fluorescence, is used to quantify the amount of DNA amplification. Using specially developed primers, PCR can be designed to amplify specific DNA sequences, thereby providing high diagnostic specificity. This helps to distinguish between different infections strains to coordinate appropriate treatment. Fluorescence signal characterization shows the initial amount in the sample before amplification. From the early stages of infection, qPCR can identify and characterize low infection rates. Theoretically, a well-designed PCR reaction with little or no non-specific binding can detect the presence of DNA molecules in the reaction tube. However, PCR analysis poses a major logistical challenge for junior clinics because it uses methods that require equipment such as a clean bench, thermal cycler and detection module or a Real Time PCR instrument that can run into extremely huge costs. In addition, the cost of maintaining such equipment and structures increases.

One reason why nucleic acid amplification appears to be cumbersome is the need for initial extraction of the pathogen's nucleic acid, amplification of the sequence of interest in the nucleic acid, and then analysis of products obtained. Less cumbersome and more straightforward non-PCR based isothermal amplification techniques provide a suitable alternative to PCR based techniques. These methods operate at a constant temperature and do not require changes in the process, which limits the use of thermal cycles. In addition, these methods can be used to record real-time amplification measurements and detect amplification products by measuring fluorescence, turbidity or visually inspecting colour changes. Due to their low power consumption and simple, it can be integrated into simple and compact systems, and hence isothermal amplification methods provide a better alternative to the traditional PCR methods especially for POC devices.

One such method of isothermal amplification relies on the use of multiple primers, to initiate the polymerase-driven extension of the gene sequence. The amplification is facilitated and enhanced by the formation of structures created by the primers/reagents. The sequence is then amplified and process is repeated, to eventually generate copious amounts of double-stranded DNA product. In an RNA-based target, a reverse transcriptase enzyme can be incorporated directly into the reaction. Single DNA detection and RNA analysis shorten the operation time for target amplification. The detection of amplicons from reaction is done either using intercalating dyes, added either to the end-point reaction, or added prior to the reaction or by using fluorescent dye-labelled probes to measure the real-time increase in fluorescence.

With respect to molecular detection, isothermal amplification offers a highly sensitive and rapid alternative to classic PCR and higher specificity than the rapid antigen tests. This method of amplification does not require any complicated equipment because the reaction is kept at the same temperature. These methods are robust when detecting directly from sources such as blood, urine, stool various media components and insects, which means there was no need for additional DNA/RNA extraction equipment. Isothermal amplification methods and technologies hold a large stake, owing to characteristics such as simplicity, cost-effectiveness, robustness, sensitivity and specificity.

For instance, In India, the main amplification-based laboratory diagnostic modality for SARS-CoV-2 detection is the Real Time Reverse Transcriptase Polymerase chain reaction (RT-PCR). Real-time RT-PCR is a special version of PCR, which can directly amplify RNA sample and removes the need of post amplification end point analysis and the amplification which can be monitored in real time. It has high precision with increased sensitivity and specificity. For positive and negative controls, the results are verified for every PCR run, thereby minimizing the possibility of false positive and false negative results. High-throughput laboratories generally process samples in 96 to 384 well plates at a time, which means this test require special central processing labs where testing can be done in batches.

This type of molecular diagnostics testing is often practiced in centralized laboratories, which require trained personnel with technical expertise to perform and interpret the test, regulated infrastructure that can transport and store reagents in controlled environment, and expensive, high throughput instrumentation. The regulators require the centralized laboratory to be a Biosafety level 2 (BSL-2) type of laboratory to perform this test, which means this test is not for basic/tertiary laboratories. These laboratories need to be designed with proper workflow to avoid the risk of occupational health hazards and sample contamination that may affect the results and cause false positive results. Not only this, the sensitivity and specificity vary based on the kits which means the results might vary and depend on subjective error due to human interference. Processing test samples typically include processing all samples collected during a time period (e.g., a day) in one large run that requires multiple steps, resulting in a turn-around time of many hours to days after the sample is collected and that makes it very time consuming. This not only takes more turn-around time from sample collection to final detection, but also requires handling of the clinical and infectious sample at various stages of the process. High device costs (one device is approximately upwards of Rs. Fifteen lakhs) and consumables along with various sophisticated equipment involved in extraction and lengthy PCR process despite its accuracy make RT-PCR tests a disadvantage. In country like India with huge population, during the COVID-19 pandemic labs conducting these tests were just in few hundreds. Despite the efforts and urgency, after one year of pandemic, the number of labs were scaled to only about 1500 all over the country. In times when there was sudden surge in number of cases, the time taken from sample collection to report ran upto 4 days. Thus, these tests despite being highly accurate, have limitations in terms of scalability and are not suitable for point of care settings.

Apart from RT-PCR based tests, Cartridge based nucleic acid amplification (CBNAAT) and Truenat technology-based tests are also available. These microfluidic based laboratory kits and molecular diagnostics provide flexibility (e.g., the ability to test for multiple different indications). However, such technology and equipment cannot be easily used by untrained users at POC or at home. CBNAAT requires proper temperature control as well as annual calibration of instrument, proper and adequate sample collection and transportation. Ongoing mutations within the target sequence of SARS-CoV-2 genes can alter the configuration of binding sites for primer leading to the failure of the amplification process. The other test, Truenat requires specialized vial to transport the sample, only one sample can be processed by one Truenat machine at a time resulting in lower throughput. More so, these microfluid based tests require cartridge to run which adds to the cost per test as cartridges are replaced after certain usage. The device costs of machines to run these tests also are extremely high (upwards of Rs. Ten lakhs each) and thus out of reach for low resource labs and hospitals. Each test for the lab can cost upwards of few thousand rupees to fifteen hundred rupees. Thus, these devices and methods are complicated to use and include expensive, sophisticated components which makes them an unviable option for low resource health care centres. Therefore, in a decentralized setting the laboratory methods and equipment such as (e.g., POC or in-home use) cartridge-based tests are expensive, inefficient, and are not a viable option for low resource point of care settings.

One of the key phases in performing POC in-home tests is its interpretation. Some molecular diagnostic tests rely on the user to visually inspect a detection window or strip to determine whether a colour change occurred thereby indicating a positive result. Other known tests and methods rely on the user to compare two different portions (e.g., strips) to make a determination regarding whether the test is positive or negative. Although in some instances such known methods can produce acceptable results, in instances when the device does not behave as intended, the results can be misinterpreted.

Another disadvantage of existing POC or in-home tests is the guidance on tracking, tracing and follow-up care. Results have to be monitored by authorities at earliest, so that preventive and proactive actions can be taken. By their very nature, such known tests and methods are usually conducted in a decentralized location by untrained users. Therefore, follow-up care is often only received if the user proactively contacts a healthcare provider and appropriate authorities/centralised database to track the spread of disease.

Having access to low-cost, easy to manufacture and low-maintenance detection equipment at a primary level such as doctor and health care centres, tertiary laboratories, clinics enables rapid on-site diagnosis which brings a great benefit to community health. The infection can be treated before it causes further harm to the patient. The clinic can make better use of its limited medical resources and if necessary, the authorities can set up quarantine areas to quickly control infectious diseases. In country like India where there are more than one lakh tertiary labs, approximately two lakh primary, community and sub-healthcare centres and about seventy thousand public and private hospitals, there are only a few thousands of centralized labs (BSL-2) for highly sensitive COVID-19 testing. This presents an extremely huge gap and urgent need for developing a testing platform exclusively for low resource point of care (POC)settings that can cater to thousands of healthcare centres and hospitals. Such testing platform with high sensitivity &mobility is also required at sensitive and inaccessible places such as borders forces, on merchant and navy ships, remote places and villages where facilities are bare minimum.

Therefore, there is an urgent need for the development and application of a POC diagnosis in this field, and to help control and manage infections. Researchers all over the world are working on the advancement of biosensors, microfluidics, bio analysis platforms, analysis formats, and Lab-on-a-chip technology. Huge potential is also observed in the smartphone-based biosensors that can be used in POC diagnostic devices.

Hence, it is an object of present invention is to provide an economical (at a small fraction of device cost and small fraction of per test cost compared with closest highly sensitive microfluidic POC tests), rapid and portable testing platform with high sensitivity for detection of pathogens.

It is another object of the present invention is to provide a simple method for detection of pathogens using the detection device of the invention.

### SUMMARY OF INVENTION

In view of above objects, the present invention provides a portable device for economical and rapid photodetection of pathogens in a biological sample.

The invention further provides a method for economical and rapid detection of pathogens in a biological sample using the portable device of the present invention. The clinical sample collection procedure is simplified by using direct collection of samples from saliva samples or other biological samples such as but not limited to nasal, throat or nasopharyngeal swabs. The method consists of simplified process for inactivation of infectious samples followed by lysis of the pathogen to release the nucleic acid for molecular detection. The detection of the target nucleic acid is then done using amplification at isothermal or thermal cycling conditions followed by an endpoint fluorescence detection using a portable molecular diagnostic test device.

### BRIEF DESCRIPTION OF DRAWINGS

FIG. 1 illustrates a schematic diagram of the device of the invention.

FIG. 2A illustrates the perspective view of the device of the invention according with lid closed.

FIG. 2B illustrates the perspective view of the device of the invention according with lid open.

FIG. 3 illustrates the exploded view of the device of the invention according to one of the embodiments of the invention.

FIG. 4 illustrates the exploded view of the detection module in accordance with one of the embodiments of the invention.

FIGS. 5A & 5B illustrates the front and rare view of the device of the invention according to one of the embodiments.

FIG. 6 illustrates perspective view of the device (2) of the invention in accordance with one of the embodiments of the invention.

FIG. 7 illustrates exploded view of device (2).

FIG. 8 illustrates the flowchart for the detection of presence of a target nucleic acid in a sample using the device of the invention.

FIG. 9 illustrates a process tray for preparation of a sample for detection using the device of the invention.

FIG. 10 illustrates a process mapping sheet (1000) which is designed for mapping the progress of the process for detection of nucleic acid.

FIGS. 11A and 11B illustrate the graphical user interface displayed on the display screen (82).

FIG. 12 illustrates the actual image of the apparatus according to the invention.

### DETAILED DESCRIPTION OF DRAWINGS

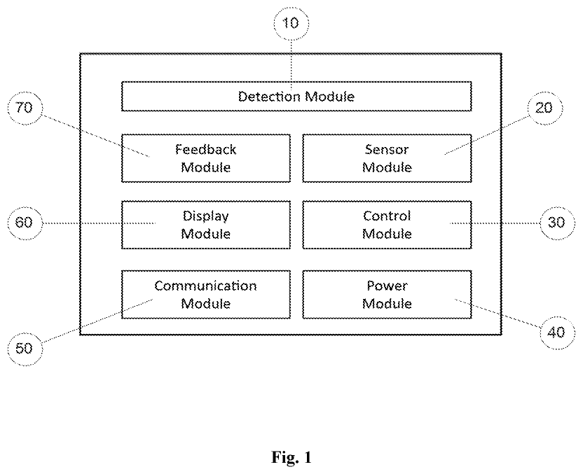

FIG. 1 illustrates a schematic diagram of the device of the invention. Accordingly, the device of the invention comprises of a Detection module (10), a Sensor module (20), a Control module (30), a Power module (40), a communication module (50), a display module (60) and a Feedback module (70). According to one of the embodiments of the invention, the Detection module (10), the Sensor module (20), the Control module (30), the Power module (40), the Communication module (50) and the display module (60) can be integrated within a housing (90), such that the molecular diagnostic test device is a portable device.

The detection module (10) is configured to allow reaction of a biological sample in which one or more reagents cause production of one or more assay signals to indicate presence of the target polynucleotide sequence. Particularly, the detection module (10) defines a detection volume within which the biological sample and one or more reagents react. The reaction can be performed by combining (e.g., mixing) the reagent and the biological sample input within a process tray (900), by introducing each of the reagent and the biological sample into the reaction module (not shown). In one of the embodiments, the detection module (10) can include one or more detection chambers in which different reagents or probes can be combined or reacted with the biological sample to produce a series of assay signals.

The sensor module (20) is configured for generating assay signals. The sensor module (20) if further configured to receive one or more light signals and produce a sensor signal associated with the light signal.

The Control module (30) comprises of a processor/controller configured to perform the processes in accordance with this invention. The processor can be configured to run and/or execute application modules, processes and/or functions associated with the detection module (10) to run and/or execute the communication module (50), and/or any of the other modules described herein. The processor may be configured to retrieve data and/or write data to memory.

The Power module (40) can be powered using an external power bank or a battery set (with rating of 5V 1 A), allowing the diagnostic test(s) to be run without A/C power, and at a suitable location (e.g., outside of a laboratory and/or at any suitable “point of care”). Thus, making the molecular diagnostic test device portable.

The Communication module (50) gives the palm sized molecular diagnostic test device an ability to communicate to a centralized web server via external wired or wireless communication devices, such as but not limited to, smart phones, tablets, etc. which may be configured to further process the data for easier analysis. The configuration of the communication devices could be achieved by installing a software which has been designed to analyse the data communicated by the communication module (50). The software may be delivered as an app in the communication device or a programme designed to be installed on a computer. The app may be delivered to device as an android application package or iOS App store package. In one of the embodiments, the application may be delivers as a program to be installed on a computer, akin to Microsoft installer package.

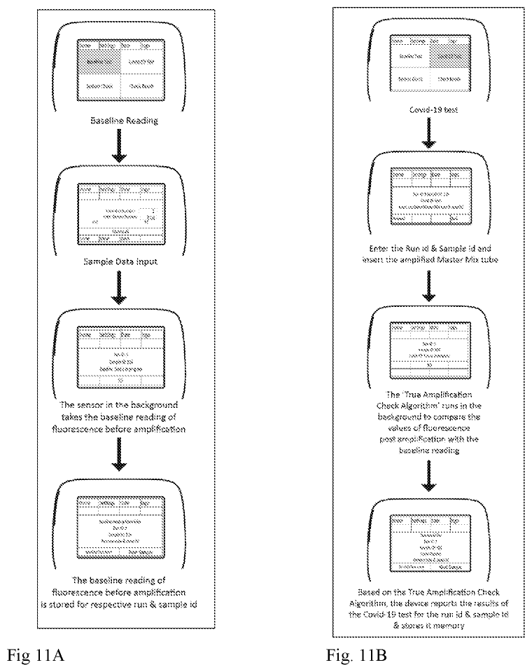

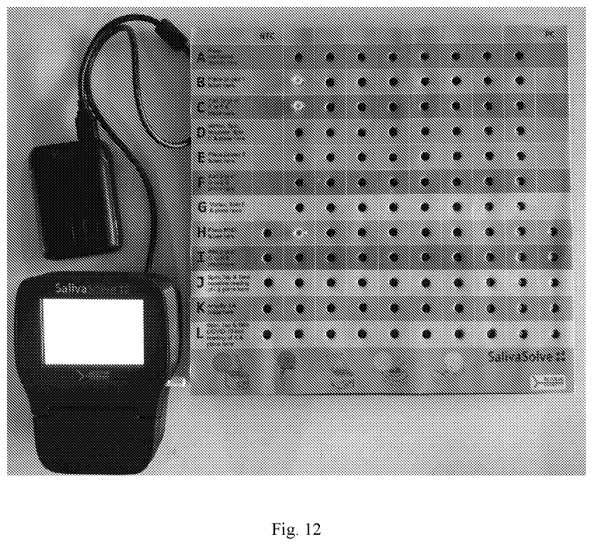

The display module (60) is configured to take input as well provide output to the lab technician, giving a visual cue about the whole process. The display module (60) contains a display screen (82) which may be configured to display the result along with audio or sound through application stored in the memory. In some embodiments, the output device can also include any combination of visual, audible, haptic, and/or wireless output mechanism. In a preferred embodiment the screen (82) is a touch screen upon which a series of graphical user interface elements (e.g., windows, icons, input prompts, graphical buttons, data displays, notification, or the like) can be displayed, as illustrated in FIGS. 11A and 11B.

The feedback module (70) is configured for storing & publishing the data on the device for user reference. The feedback module (70) instructs the display module (60) to display the run, sample & result of the test.

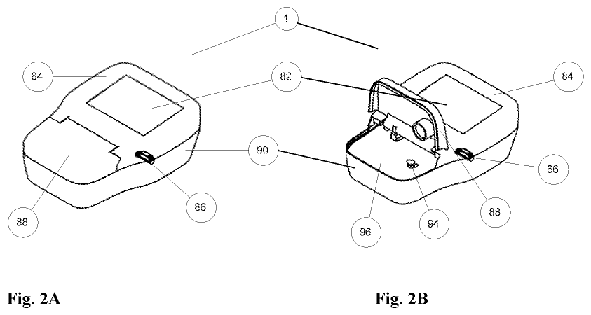

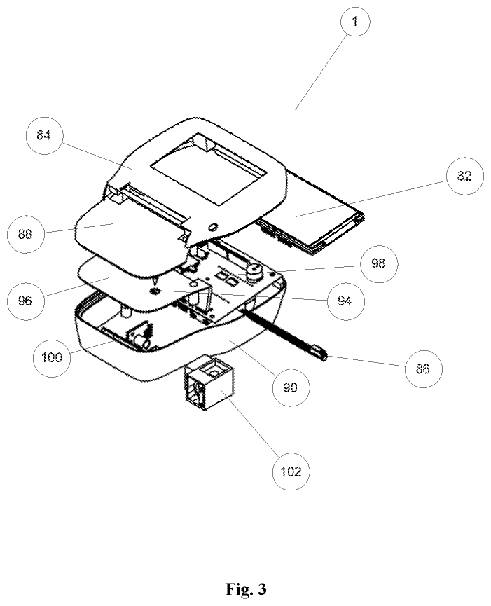

FIG. 2A illustrates the perspective view of the device (1) of the invention in accordance with one of the embodiments with the lid (88) closed while FIG. 2B illustrates the device (1) of the current embodiment with lid (88) open. Further, the FIG. 3 illustrates an exploded view of the device (1) of the invention in accordance with one of the embodiments of the invention.

Accordingly, the device (1), in accordance with this embodiment, comprises of a display (82) encased in a controller board housing (84). The device (1) is further provided with a lid (88), which is in mechanical contact with the controller board housing (84). Further, a stylus (86) is provided along with the device (1) which is housed in the controller board housing (84). The control board housing (84) houses a control board (98) which is in electrical contact with the display screen (82)

A bottom case (90) is placed on the opposite side of the controller board housing (84) and forms the bottom potion of the device (1). The bottom case or housing (90) functions as a housing for other components of the device (1).

A middle tray (96) is positioned below the controller board housing (84) and at approximate middle of the height of the bottom case (90). The middle tray (96) functions to hold the components of the device (1). The middle tray houses a tube insertion hole (94) configured for holding a sample tube (104). The tube insertion hole (94) is superimposed on tube holder (102). An optical assembly (100) is aligned with the tube holder (102), such that the optical signals from the tube holder (102) are directly incident on the optical sensor of the optical assembly (100).

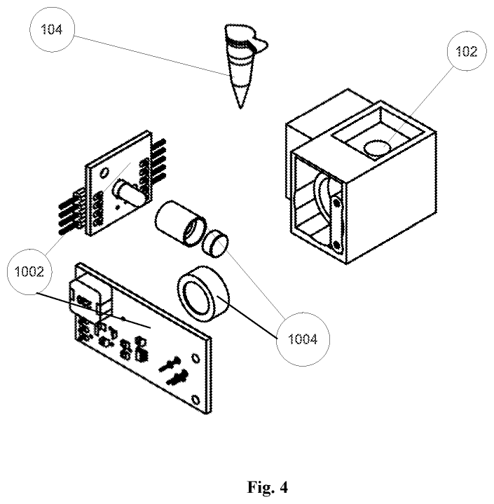

An exploded view of the optical assembly (100) is illustrated in FIG. 4. The optical assembly (100) comprises of at least one optical assembly (1002) and at least one optical lens (1004).

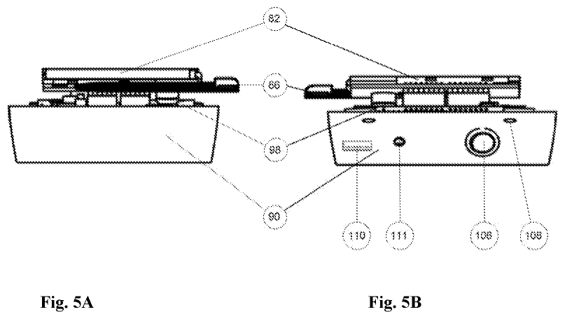

As illustrated in FIGS. 5A and 5B, the device of the invention is provided with a power socket (111) and a power on/off switch (106) on the top portion of the device. The device (1) of the invention is further provided with a USB port (110). Said USB Port (110) may function to connect the device to an external computer and/or processing device for further analysis of the data. Alternatively, the USB port can function as a charging port for charging a battery, if included in the device. The device may further be provided with mounting holes (108) for wall mounting of the device.

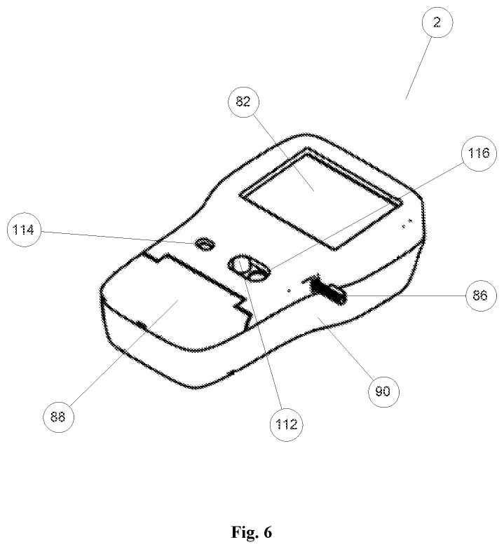

FIG. 6 illustrates a device (2) according to one of the embodiments of the invention. The device (2) further comprises a shutter (112), a shutter open/close indicator (114) and a view hole (116) in addition to the components of device (1).

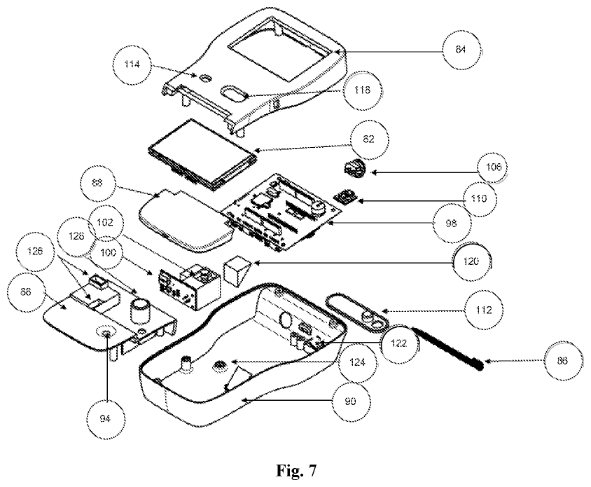

FIG. 7 illustrates one more embodiment of the invention whereby the device, in addition to the device as described in FIG. 2A further comprises of view hole (118), a prism (120) located just beneath the view hole (118), a shutter (112) interposed between the view hole (118) and prism (120). The arrangement containing the shutter (112), the view hole (118) and the prism (120) is designed to facilitate quick visual inspection of the progress of the detection process. The device (2) further comprises a view tunnel (128).

Both the devices (1) and (2) are provided with mounting holes (124) on the base of housing (90). The devices (1) and (2) are further provided with a USB wire claim 122) placed in electronic connection with USB port (110).

Both the devices (1) and (2) further comprise a lid open sensor (126)

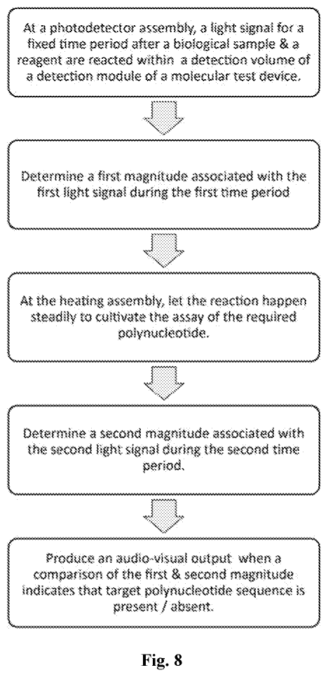

FIG. 8 illustrates a flowchart for detection of presence of a target nucleic acid in a sample.

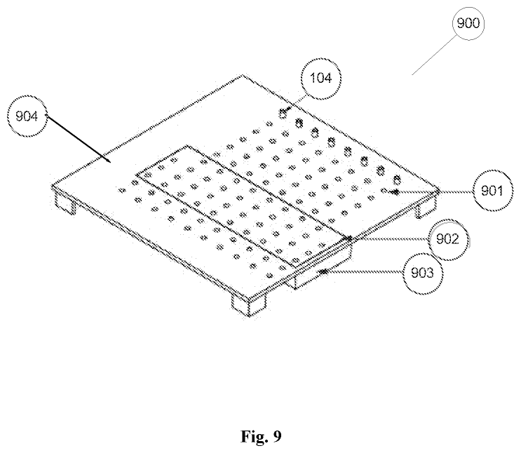

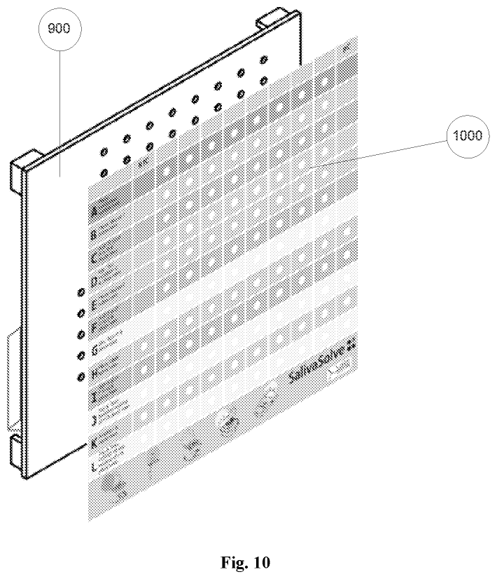

FIG. 9 illustrates a process tray (900) which can be used for sample preparation. The process tray (900) consists of metal or plastic block (904) which has been demarcated with insertion holes (901). The insertion holes (901) are configured to hold the tubes (104) containing the reagents, enzymes, master mix and target sequences. The process tray (900) is further, optionally, provided with a chiller (902) cooled by a refrigerant tray (903).

### DETAILED DESCRIPTION OF INVENTION

The present invention describes a portable system and a device for economic and rapid collection and photodetection of pathogens in a biological sample and a method thereof.

Unless otherwise specified, the terms apparatus, diagnostic apparatus, diagnostic detection system, diagnostic test, diagnostic test system, diagnostic detection test unit, and variants thereof, can be interchangeably used.

Terms and symbols of nucleic acid chemistry, biochemistry, genetics, and molecular biology used herein follow those of standard treatises and texts in the field and the like.

The term “pathogen” may refer to a microorganism, such as one or more bacteria, fungi, protozoa, viruses capable of causing disease. In some embodiments, the pathogen is multicellular (e.g., a worm or other parasite).

As used herein, a “biological sample” refers to any tissue or fluid obtained from an organism (e.g., a subject, e.g., a human or animal) containing polynucleotide's (e.g. (e.g., DNA or RNA), the equipment described in this document can be used for amplification and/or detection. In some embodiments, any of the devices and methods described herein can be performed on several different types of samples. The primary sample used in the invention includes saliva, but however other such sample types can optionally also be embodied for, example a buccal smear, stool, sputum, nasal wash, nasal aspirate, throat swab, vaginal swab, penile-meatal swab, sample bronchial lavage, blood, blood cells (e.g., white blood cells), fine needle biopsy samples, peritoneal fluid, visceral fluid, pleural fluid, a urine sample, rectal swab sample and/or pharyngeal swab sample, or cells there from. Other biological samples useful in the present invention include tumour samples (e.g., biopsies) and blood samples. The term “biological sample” also refers to a portion of the resulting tissue or purified fluid (e.g., that has been filtered, lysed, prepared, amplified or reacted) in connection with the diagnostic methods described herein. Thus, a biological sample can refer to a raw sample (e.g., a raw blood sample) obtained from a patient, as well as a portion of the raw sample that has been “prepared” for use, reacted, or amplified in any of the devices or methods described herein.

The term “nucleic acid molecule,” “nucleic acid,” or “polynucleotide” may be used interchangeably herein. Unless otherwise specified, deoxyribonucleic acid (DNA) or ribonucleic acid (RNA) includes known analogues or combinations thereof. The nucleic acid molecules described herein can be obtained from any nucleic acid source. The molecules of nucleic can be single-stranded or double-stranded. In some cases, the nucleic acid molecules are DNA. The DNA can be mitochondrial DNA, complementary DNA (cDNA), or genomic DNA. In some cases, the nucleic acid molecule is genomic DNA (gDNA). The DNA can be cosmid DNA, plasmid DNA, yeast artificial chromosome (YAC) or bacterial artificial chromosome (BAC). The DNA can be copied from one or more chromosomes. In some cases, the nucleic acid molecules include, but are not limited to, mRNAs, tRNAs, snRNAs, rRNAs, retroviruses, small non-codingRNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long ncNAs. Bacterial resistance may be conferred by plasmids or phage and in such cases the polynucleotide may be the plasmid or the phage genome. In some embodiments, “a polynucleotide associated with a target organism” refers to two or more polynucleotides. The source of nucleic acid used in the devices, methods, and compositions described herein may be a biological sample containing nucleic acid.

“Target nucleic acid sequences” or “target polynucleotides (or polynucleotide sequences)” include genomic nucleic acids of a particular organism. Such target nucleic acid sequences may be single stranded or double stranded and may include a sense strand and/or an antisense strand. These target nucleic acid sequences can be deoxyribonucleic acid (“DNA”) or a ribonucleic acid (“RNA”) either of viral, bacterial, parasitic or fungal origin. Hence, the term “viral nucleic acid” is also used occasionally throughout the current specification interchangeably to indicate target nucleic acid.

In some embodiments, the amplification/heating process may be conducted by a completely different module and may be with or separate from the detection module. The target nucleic acid or polynucleotide sequences may be amplified using methods known to those of skill in the art. Such techniques include the use of polymerases, primers and nucleotides. “Amplifying” includes the production of copies of a nucleic acid molecule via repeated rounds of primed enzymatic synthesis.

Amplification methods may comprise contacting a nucleic acid with one or more primers that they hybridize specifically to nucleic acids under conditions that promote hybridization and chain extension. Exemplary methods for amplifying nucleic acids include the polymerase chain reaction (PCR), anchor PCR, RACE PCR, ligation chain reaction (LCR), self-sustained sequence replication, transcriptional amplification system, Q-Beta Replicase, recursive PCR; Loop-Mediated Isothermal Amplification, Recombinase Polymerase Amplification, Helicase-dependent Amplification, Rolling Circle Amplification or any other nucleic acid amplification method using techniques well known to those of skill in the art. In some embodiments, the methods disclosed herein utilize reverse transcription isothermal amplification.

“Isothermal amplification of nucleic acid” and its other derivative methods and techniques provide detection of a nucleic acid target sequence in a rationalized, exponential manner, and are not restricted by the constraint of thermal cycling. Isothermal amplification methods provide a rapid, sensitive, specific, simpler and less expensive procedure for detecting nucleic acid from samples. The isothermal methods rely on an alternative approach to enable primer binding and initiation of the amplification reaction: a polymerase with strand-displacement activity. After the reaction starts, the polymerase should also separate the strands that are still hybridizing to the target sequence. Within isothermal amplification methods differ from one another in features such as the number of primers and enzymes, the amplification temperature, and the type of template used.

In some embodiments, a detection module includes one or more probes designed to bind to an amplicon associated with the target polynucleotide sequence. The term “probe” as used herein refers to fluorescent dye-labelled oligonucleotide used to capture a target amplicon.

A probe according to the present disclosure may be referred to as a hybridization probe which is a fragment of DNA or RNA of variable length which is used in DNA or RNA samples to detect the presence of nucleotide sequences (the target amplicon) that are complementary or substantially complementary to the sequence in the probe. The probe thereby hybridizes to single stranded nucleic acid (DNA or RNA) whose base sequence allows probe-target base pairing due to complementarity between the probe and target amplicon.

In an embodiment, the present invention provides a portable device for detection of pathogens in a clinical sample. Accordingly, the portable device comprises of a housing, a power module, a controller, a display module, a detection module, and a wireless communication module. The power module, the controller module, the display module, detection module & the wireless communication module are integrated within the housing, such that the molecular diagnostic test device is a portable device. The power module can also be powered using an external power bank or a battery set (with rating of 5V 1 A), allowing the diagnostic test(s) to be run without A/C power, and at a suitable location (e.g., outside of a laboratory and/or at any suitable “point of care”). Thus, making the molecular diagnostic test device portable. The wireless communication module gives the palm sized molecular diagnostic test device an ability to communicate to a centralized web server via external wireless communication devices (like smart phones, tablets, etc.), which further process the data for easier analysis.

In accordance with above embodiment, the portable molecular diagnostic test device includes a housing (90), a detection module (10), and a control module (30). The detection module (10) detects the amplification of the sample to produce a signal that indicates a presence of target amplicon with the input sample. The detection module & control module are integrated within the housing such that the molecular diagnostic test device is a portable device.

In some embodiments, the apparatus is configured for a disposable, portable, inexpensive, molecular diagnostic approach. The apparatus can include one or more modules configured to perform high quality molecular diagnostic tests, including, but not limited to, sample preparation, nucleic acid amplification, and detection.

Further, the apparatus can be powered using an external power bank, or a 5V 1 A battery set, allowing the diagnostic test(s) to be run without A/C power, and at a suitable location (e.g., outside of a laboratory and/or at any suitable “point of care”). For example, in some embodiments, the power module can be operated by a small battery (e.g., a 5V battery) and can include a controller to control the timing and/or magnitude of power draw to accommodate the capacity of the battery. In other embodiments, the apparatus can include any number of features such as safety locks, configured to minimize the chances of user error.

In some embodiments, a hand-held molecular diagnostic test device includes a housing, a controller module, detection module, power module and wireless communication module. The wireless communication module is configured to receive an output from the controller module and forward it to an external communication module (e.g., smartphone, tablet, etc.), which can further process the data and upload it to a centralized server. In some embodiments, an apparatus includes a display module which can be configured to take input as well as provide output to the lab technician, giving a visual cue about the whole process.

In some embodiments, as apparatus can include a heater module, which could be configured to control the amplification process to amplify the target nucleic acid strain in the sample. The heater assembly can be defined into at least 2 heating zones, both being set at different temperatures one for amplification and other for sample inactivation. Both the heater assembly can be configured to maintain a temperature in each zone. In some embodiments, a diagnostic apparatus is configured for a portable, low-cost, molecular diagnostic approach to include one or more modules configured to perform high quality molecular diagnostic tests, including, but not limited to, sample preparation, nucleic acid amplification using thermal cycler (e.g., via polymerase chain reaction, isothermal amplification, or the like), sample mixing module and detection module.

Detection can occur, in some embodiments, through fluorescence intensity detection or through a signal that indicates a presence of target amplicon. Accordingly, to detect hybridization of the target amplicon to the probe, the target amplicon is tagged (or “labelled”) with a molecular marker or label, for example a fluorescent marker or any enzyme capable of generating a coloured or fluorescent signal in the presence of an appropriate enzyme substrate or coupled with a quencher system that may be a separate oligonucleotide or a dual-labelled probe with fluorescent-dye and quencher tagged to the same oligonucleotide.

Visually detectable markers suitable for use in the devices and methods of the disclosure include various enzymes, prosthetic groups, fluorescent markers, luminescent markers, bioluminescent markers and so on. Examples of appropriate fluorescent groups include, but are not limited to, yellow fluorescent protein (YFP), green fluorescence protein (GFP), cyan fluorescence protein (CFP) and the like. Examples of quencher moieties include, but are not limited to, Black Hole Quencher 1 (BHQ1), Black Hole Quencher 2 (BHQ2) and the like.

In some embodiments, the detection device of the invention is termed as “SalivaSolve® Device” (and any of the devices shown and described herein) can be a National Accreditation Board for Testing and Calibration Laboratories (NABL)-waived device and/or can operate in accordance with methods that are NABL waived. Similarly stated, in some embodiments, the device SalivaSolve® (and any of the other devices shown and described herein) configured to work in a relatively simple way, and the results show sufficient accuracy that represents a limited possibility of abuse and/or limited risk of injury if used improperly. In some embodiments, the device SalivaSolve® (and any of the other devices shown and described herein), can be operated according to the method by users with minimal (or no) scientific training, in accordance with methods that do not require the user to understand and/or simply and/or automatically perform certain work steps.

In some embodiments, the devices described in this document are standalone devices, which may not include all necessary substances, mechanisms, and sub-assemblies to perform any of the molecular diagnostic tests described herein. Such stand-alone devices may require any external instrument to manipulate the biological samples, and, in some embodiments, may only require connection to a power source (e.g., a connection to an A/C power source, coupling to a battery, or power bank or the like) to perform the methods described in this document. For example, the device described herein requires an external instrument to heat the sample for amplification, a mixer to agitate or mix the sample, or the like. Rather, the embodiment described herein is only used to detect the presence of target polynucleotide sequence after it has undertaken the process of incubation/heating and mixing with reagents via nucleic amplification. The biological sample is then used as input in the embodiment described herein the device that can be actuated to perform the molecular diagnostic tests described herein.

In an embodiment, the portable molecular diagnostic test device includes a housing, a power module, a controller/processor, a display module, a detection module, optic/light sensor module, and a wireless communication module. The power module, the controller/processor module, the display module, detection module, optic/light sensor module & the wireless communication module are integrated within the housing, such that the molecular diagnostic test device (“detection module”) is a portable device. The power module can also be powered using an external power bank or a battery set (with rating of 5V 1 A), allowing the diagnostic test(s) to be run without A/C power, and at a suitable location (e.g., outside of a laboratory and/or at any suitable “point of care”). The wireless communication module enables portable molecular diagnostic equipment to transmit results of the diagnostic test to an app on an external wireless communication device (such smart phones, tablets, etc.), which may then can communicate to a centralized web server to further process the data for central records and analysis.

In one more embodiment, the portable molecular diagnostic test device includes a housing, a detection module, optic/light sensor module, and a controller and processor module. The detection module detects the amplification of the sample to produce a signal that indicates through optic/light sensor and reading module the presence of target amplicon with the input biological sample. The detection module & controller module are integrated within the housing such that the molecular diagnostic test device is a portable device. The diagnostic apparatus may also include one or more modules configured to perform high quality molecular diagnostic tests, including, but not limited to, a process tray (900), nucleic acid amplification/heating (Incubation)module, mixing module and detection module. A target nucleic acid in the target entity can be amplified with a heating step using primers, reagents and thermal cycler to yield a greater number of copies of the target nucleic acid sequence for detection. Detection can occur, in some embodiments, through fluorescence detection or a signal that indicates a presence of target amplicon.

In some embodiments, sample preparation can be performed by isolating pathogens/subjects and removing unwanted amplification inhibitors. The target entity can be examined later to release target. A target nucleic acid (e.g., target polynucleotide sequence) in the target entity can be amplified with a polymerase undergoing temperature cycling or via an isothermal incubation to obtain more copies of the target nucleic acid sequence for detection.

In some embodiments, the process tray (900) defines a biological sample input (SI₁) volume that receives a biological sample. The biological SI₁ sample can be transported to the detection device manually or through the sample transfer device after undergoing amplification and mixing with reagents and primers for detection of target amplicon. The sample transfer device can be any suitable device, such as a pipette or other mechanism configured that can be used to aspirate or withdraw the sample from a sample cup, container or the like, and then deliver a desired amount of the sample via the opening in detection device. The process tray (900) may include any of the components described herein for manipulating biological sample SI₁ for further diagnostic testing and/or to produce a solution for detection of a nucleic acid.

In some embodiments, the device of the invention may include a heating system which can include one or more heaters and cooling modules through one or more chambers within which the biological sample SI′ can be manipulated, after mixing with one or more certain on-board reagents using a mixing device module. In some embodiments, the heating system can function merely as a sample holding or mixing chamber. For example, in some embodiments, the heating system may include the desired amplification reagents to facilitate a desired amplification according to any of the methods described herein.

In yet other embodiments, the process tray (900) can perform all or any series of operations a) receiving the biological sample SI₁, b) mixing the biological sample SI₁ with desired reagents to get biological sample SI₂, c) performing incubation, heating & mixing operations to release target RNA from the biological sampleSI₂, to generate biological sample SI₃ d) adding reagents and primers to the resulting solution SI₃ to generate SI₄. Thus, in some embodiments, the process tray (900) enables an efficient, sample input preparation SI₄ to be performed within a single environment or module that is used as input in the detection device module.

The process tray (900) can also incorporate a reaction module that defines a reaction volume and includes a heater. The reaction volume can be formed by any suitable structure having a certain volume or a volume range within which the input solution can flow and/or be reacted to produce a solution that is conveyed into the detection module. Thus, the reaction module can function as an amplification module, a lysis module, or any other module within which a reaction can occur to facilitate detection of the target polynucleotide sequence. In some embodiments, the reaction module can amplify the target nucleic acid molecules therein to produce an output detection solution that contains a target amplicon (or multiple target amplicons) to be detected.

The heater can be any suitable heater or group of heaters that can heat the input solution SI₂ or SI₃ to perform any of the amplification/incubation operations as described herein. In other embodiments, the amplification/heater module (or any of the amplification modules described herein) can be separate from the process tray (900) and can amplify the target nucleic acid molecules therein to produce an output detection solution that contains a target amplicon (or multiple target amplicons) to be detected.

The detection module described herein is configured to react the biological sample (identified as the processed Sample Input SI₄) in which one or more reagents cause production of one or more assay signals to indicate presence of the target polynucleotide sequence. The biological sample is identified as a portion (i.e., SI₄) of the initial biological sample SI₁ that has been processed, reacted or prepared within the process tray (900), the reaction module, amplification/incubation/heating module, mixing module together as one diagnostic apparatus or as separate standalone apparatus. The portion of the biological sample input SI₄ is used in the detection module. As described herein presence of the target polynucleotide sequence can indicate the presence of a target organism. Specifically, the detection module defines a detection volume within which the biological sample and one or more reagents reacted. The reaction can be performed by combining (e.g., mixing) the reagent and the biological sample input within the process tray (900), by introducing each of the reagent and the biological sample. In other embodiments, the detection module can include one or more detection chambers to detect the presence of target polynucleotide sequence in in the biological sample by detecting the series of assay signals produced.

Amplification may be performed with any suitable reagents. The term “reagent” includes any substance that is used in connection with any of the reactions described herein. Reagent may include but not limited to, template nucleic acid (e.g. DNA or RNA), primers, probes, buffers, enzymes, wash, replication catalysing enzyme (e.g. DNA polymerase, RNA polymerase), nucleotides, salts, non-ionic detergents, additives that reduce stability of double bonded DNA, facilitate specific region amplification, reduce amplification inhibitors, reduce secondary structure, stabilise polymerase, reduce potential DNA-RNA mismatch, improve hybridization stringency, recombinant eliminating contamination, causing highly specific cleaving actions, remove gDNA, cleaving specific strands in RNA-DNA hybrids, ionic cryo-protectants, surfactants, non-ionic surfactants, pH-buffering agents, etc. A reagent can include a mixture of one or more constituents (active and/or inert constituents) regardless of their state of matter (solid, liquid or gas) and can be either in a mixed state, in an unmixed state and/or in a partially mixed state.

In some embodiments, an amplification reaction is any reaction in which nucleic acid replication occurs repeatedly over time to form multiple copies of target nucleic acid molecule (e.g., DNA, RNA). Amplification reaction is achieved at isothermal temperature or via thermal cycling. The nucleic acid amplification methodology mentioned herein relies on thermal transfer steps to amplify target nucleic acids.

In one for the embodiment, the present invention provides reagents (e.g., primers, buffers, salts, nucleic acid targets, etc.) and methods for the amplification of nucleic acid. The present invention utilizes two lysis solutions, developed in-house, comprising of reagents, enzymes, etc, and for the purpose of identification having brand name SkipEX 1 and SkipEX 2 and together SkipEX, a two-tube lysis-based enhancement to increase the efficiency of RNA/DNA detection.

The reagent SkipEX is formulated to facilitate production of the assay signal or enhance the assay signal indicating the presence of the target polynucleotide sequence. The reagent SkipEX can be stored within the process tray (900) or stored within an enzyme/reagent module or any other module separately. For example, in some embodiments, the reagent can be in a liquid state and can be stored in a sealed container while in other embodiments; the reagent can be in a solid state. In some embodiments, the molecular diagnostic test apparatus can include two or more reagents to facilitate production of the assay signal or to enhance the signal that indicates a presence of the target polynucleotide sequence (e.g., within the solution SI₄).

The assay signal(s) can be any signal indicating the presence of the target polynucleotide sequence. For example, in some embodiments, the assay signals can be colorimetric signals produced by the substrate where the detection of the assay signal is accomplished with separate light source that is passed through or onto the detection module (and the colorimetric assay signals) to determine the presence of the colorimetric assay signal. Alternatively, the assay signals can be chemical luminescence signals produced by luminescence reaction where the assay signal itself can be detected by the optic/light sensor that measure the light intensity. In yet other embodiments, the assay signals can be fluorescence signals produced when an excitation light source excites the biological solution SI₄ in the detection module.

The electronic detection system can be coupled to or is within the housing in the detection module device. The electronic detection system can perform electronic detection of the assay signal and produce an electronic output, as described herein. The electronic detection system includes a processor and controller, a memory, an optic/light sensor, and an output device. The electronic detection system also includes one or more applications or modules that are implemented in at least one of the memory or the processor to facilitate communication module.

The processor/controller and each processor described in this document can be any suitable processor that performs the processes described in this document. The processor can be configured to run and/or execute application modules, processes and/or functions associated with the detection device module to run and/or execute the communication module, the sensor module, and/or any of the other modules described herein. The processor may be configured to retrieve data and/or write data to memory.

The memory in the processor can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, or any other digital data storage device. In some embodiments, the memory stores instructions that cause the processor to execute modules, procedures, and/or functions associated with the optic/light sensor module. For example, the memory can store instructions to cause the processor to execute any of the application modules described herein, and perform the methods associated therewith or store information, such as one or more thresholds or ranges to be used in the methods of detection described herein.

The sensor in the detection module can be any suitable switch, optical/light input sensors, temperature sensor, chemical sensor, and/or any other suitable sensor configured to receive one or more light signals and produce a sensor signal associated with the light signal. In some embodiments, the sensor may include one or more of the sensors described herein. In some embodiments, the optic/light sensor can be a photodetector in the detection module and that receives one or more light signals.

The output device described herein can be any suitable output device for producing one or more electronic outputs (visually, audible, haptic and/or wireless output mechanism) when the target polynucleotide sequence is determined to be present in the biological sample SI₄. The output device includes display screen that corresponds to one of the conditions to be detected by the test device. The display screen displays the test results based on an output device that includes a light output device (e.g., light-emitting diode; LED) that produces one or more light signals to convey the test results to the optic/light sensor, which then conveys it to the processor to comprehend and display the appropriate test results. Thus, the detection module displays the results once the presence of a polynucleotide sequence is detected. The display module contains a display screen shall display the result along with audio sound through a computer application stored in its memory. In some embodiments, the output device can also include any combination of visual, audible, haptic, and/or wireless output mechanism.

The detection module can be represented and described as a hardware and/or software module (stored in a memory and/or executable in a processor) as shown. The detection module is configured to receive an optic/light sensor signal and determine, based on the optic/light sensor signal, a test result (e.g., whether the assay signal is present, whether the target polynucleotide sequence is present, whether a positive control has properly produced a signal, etc.).

In some embodiments, the sensor module is configured to receive from the optic/light sensor, a sensor signal associated with the light signal for multiple times before the biological sample SI₄ is used as input for calibration. The first sensor signal using an LED light is used to for system check without a control tube in order to ensure all the modules within the detection device are working and in order. The second sensor signal is a background (or calibration check) signal with a controlled tube but without any biological sample input. The control tube can be a sequence associated with a sample that is non-pathogenic to humans, is not harmful to the environment, and is used to verify if the sensor is working and reading the light intensity correctly. Thus, if the sensor correctly reads the light intensity of the control tube and detects the light intensity reading successfully, then the proper function of the detection test device SalivaSolve® can be verified. The third sensor signal is a first light signal that is associated with the assay signal produced when the biological sample SI₄ and the enzyme/reagents SkipEX 1 & 2 and master mix have been added and introduced in the detection device but before amplification under pre-reaction stage (baseline reading). The fourth sensor signal SI₄ is the second light signal that is associated with the assay signal produced when the biological sample SI₄ and the reagents are reacted (heated) and amplified in the thermal cycler/heating/reaction module and has been introduced into the detection device for detection of the target polynucleotide sequence. The sensor module with the help of optic/light sensor reads and records in the memory the value of light intensity of the assay light signal. It determines its value of light intensity and records the reading LI1 and LI2 from the third and fourth sensor signal. Based on amplification checks through light intensity changes while normalising for sample dependent background light intensities from the readings of LI1 and LI2, the detection module determines whether the target polynucleotide sequence is present in the biological sample. In this manner, the detection module can account for differences in the values of light intensities in the first light signal (LI1) and the second light signal (LI2) from assay and sensor for pre-reaction and post-reaction readings that can result from part-to-part variability (e.g., changes in the intensity of any excitation/detection light that may be present within the biological sample.

The communication module can be a hardware and/or software module (stored in memory and/or executed in the processor). The communication module is configured to receive an indication (e.g., from the sensor(s)) and/or test result information from the detection module and cause production of one or more electronic outputs associated with the test result. In some embodiments, the electronic output can also include any combination of visual, audible, haptic, and/or wireless output mechanism.

In an embodiment, the molecular diagnostic detection device SalivaSolve® performs as per the detection methods described herein. FIG. Bis a flow chart of a method of detecting the presence of a target polynucleotide sequence within a biological sample, according to an embodiment. The method includes receiving, at a photodetector assembly, a first light signal for a first time period after the enzymes/reagents are added to biological sample but not reacted (heated). The reagent is formulated to facilitate production of a first assay signal (pre-reaction stage) and a second assay signal (post-reaction stage). The first assay signal is the background (or baseline) signal before the reaction is amplified to detect the presence of the target polynucleotide sequence. The second test signal indicates whether the reference polynucleotide sequence is present post the reaction stage when the biological sample is amplified and reaction occurs. In some embodiments, the second light signal is any one selected from colorimetric signal, a chemiluminescence signal, or a fluorescence signal.

A first reading of the value of light intensity (LI1) is associated with the first light signal, i.e., the pre-reaction reading taken and stored in the memory. The first value of light intensity can be a slope (i.e., rate of change) of the first light signal during the first time period, an average value of intensity of the first light signal in the pre-reaction stage during the first time period, or a variability of the first light signal during the first time period.

The method for detection of the target polynucleotide includes receiving, at the photodetector assembly, a second light signal for a second time period after the biological sample and the reagent are reacted in the thermal cycler/heating/amplification module and placed/transferred in the detection device module within. Therefore, the second light signal is related to the presence of the target polynucleotide sequence.

A second reading of the value of light intensity (LI2) is associated with the second light signal intensity and is recorded by the optic/light sensor and memory. The second value of light intensity can be a slope (i.e., rate of change) of the second light signal during the second time period, an average value of intensity of the second light signal in the post-reaction stage during the second time period, or a variability of the second light signal during the second time period. The first value and/or second value of the light intensity can be read and recorded within the detection module using filter and optic/light sensor, assay light signals and stored in the memory. In other embodiments, the electronic system can include signal amplifiers, filter components or the like and the first value of light intensity and/or the second value of the light intensity can be determined based on an amplification checks and filtered signal associated with the first light signal and/or the second light signal. In primary embodiment it could be all of the above.

An electronic output is produced based on true amplification checks through light intensity changes using the first value of light intensity (LI1) and the second value of light intensity (LI2) while normalising for sample dependent background light intensities to indicate the presence of the target polynucleotide sequence. In some embodiments, the comparison indicates that the target polynucleotide sequence is present when a difference between the first value of light intensity and the second value of light intensity is within a predetermined value range or beyond a certain pre-determined range defined by true amplification checks. For example, in some embodiments, if difference between the average value of intensity of the first light signal (i.e., the first value LI1) and the average value of intensity of the second light signal (i.e., the second value LI2) is greater than a specific minimum value, then the target polynucleotide sequence is considered to be present. In some embodiments, if difference between the average value of intensity of the first light signal (i.e., the first value) and the average value of intensity of the second light signal (i.e., the second value) is greater than a minimum value but less than a maximum value defined by true amplification checks, then the target polynucleotide sequence is considered to be present. In some embodiments, the evaluation indicates that the target polynucleotide sequence is present when a ratio of the first value of light intensity and the second value of light intensity is within a predetermined ratio range defined by true amplification checks. For example, in some embodiments, if ratio between the average value intensity of the first light signal (i.e., the first value of light intensity LI1) and the average value intensity of the second light signal (i.e., the second value of light intensity LI2) is greater than a minimum value (e.g., fifty percent), then the target polynucleotide sequence is considered to be present. In primary embodiment it could be all of the above.

Any of the devices described herein can include an electronic system that detects the presence of the assay light or colour signals produced in the detection device module therein. Converting the light/colour change produced by the chemical reactions into a digital result removes end-user ambiguity when interpreting test results. Additionally, the computer-implemented methods described herein can be determined based on comparison to a reference signal or other signals to improve the limit of detection and accuracy of detection. In some embodiments, the electronic system or optic/light sensor circuit may include one or more light-emitting devices and one or more photo detectors and a computer-implemented module that determines a characteristic of the light associated with the detection filters or optic/light sensors of the detection module. For example, in some embodiments, computer implemented module can determine an amount of light intensity through the detection filter(s). As the detection filter(s) or filter changes colour (as a result of the reactions described above), the amount of an incident light that passes through the detection filter(s) is recorded using sensor. By detecting the change in the value of the light intensity, the optic/light sensor can produce a digital signal to the detection module that compares two values (LI1) and (LI2) using true amplification checks, which indicates the presence or absence of the target polynucleotide sequence.

The electronic detection system may include a printed circuit board and a series of light-emitting diodes (LEDs) (collectively referred to as a light assembly) and photodiodes (collectively referred to as a photo detector assembly; only one pair of LEDs and photodiodes is identified). The printed circuit board can include a processor, and/or any other electrical components necessary for the detection module and the electronic detection system (or portions thereof) to operate as desired. The electrical components may be, for example, resistors, capacitors, inductors, switches, microcontrollers, microprocessors, and/or the like. Moreover, the detection system and its components can be electrically coupled to (or form a part of) an overall electronic control system that controls operation of the entire device.

In some embodiments, the device detection module can include any suitable shielding or light noise attenuation mechanisms to reduce light other than that emitted by the desired assay signals or LED from reaching the desired photodiode. In some embodiments, the detection module can include a cover or light shroud around substantially the entire detection module to reduce the likelihood that external light will impact the electronic detection system. In this manner, the optic/light sensor can be aligned with the LED/photodiode pairs and any light shield components used to minimize the impact of external light (or light from adjacent LEDs) affecting the detection accuracy.

The input/output system (which functions as a user interface) can include any suitable components for conveying information to, and in some embodiments, receiving information from, a user. For example, in some embodiments, the input/output system can include one or more light output devices (e.g., display screen) that communicate the instructions, settings, configuration and results that can be easily seen by the user to read the device.

In other embodiments, the input/output system can produce any suitable electronic output to be read by the user. Such electronic outputs can include an audible output (e.g., produced by a speaker), a haptic (vibratory) output, a light output (e.g., as described herein), and a wireless signal.

In some embodiments, the input/output system can include a display module/monitor or screen that displays visual elements to a user. The screen can be a touch screen upon which a series of graphical user interface elements (e.g., windows, icons, input prompts, graphical buttons, data displays, notification, or the like) can be displayed. In some embodiments, the graphical user interface elements (FIGS. 11A and 11B) are produced by a user interface module. In such embodiments, the user can also enter information into the electronic system via the input/output system.

In an embodiment, the invention provides a system for detection of viral nucleic acid in a sample. The system comprises of a process tray (900) and a portable detection device (1 or 2).

The process tray (900) is designed for preparing the sample for detection of a viral nucleic acid. The process tray (900) facilitates the end user to follow 10-12 simplified steps for processing the samples from inactivation to end-point detection.

The tray has insertions to place sample and control tubes for each step of the sample processing, including insertions holes (901) for placing corresponding reagent tubes (104) and master mix tubes (104) and allows downward process flow to minimize chances of errors while handling multiple tubes. The insertion holes (901) allow the tubes (104) to be placed in such a way that it is easy to recognise the step. The fact that the tube (104) stands upright with help of insertion holes (901) allows the lab technician to identify and easy to pick up the require tubes (104), thereby reducing the risk of amplicon contamination.

Accordingly, the process tray (900) has specific colour-codes for individual steps and easy-to-understand instructions for the end user to carry out sample processing, starting with inactivation of samples, carrying out the process for enhancing the release of nucleic acids and also includes template addition and taking readings of the samples using the portable molecular diagnostic test device.

In some embodiments, the process tray (900) could be equipped with a cooling system comprising of a chiller tray (903) and aluminium block (902) to keep the tubes (104) containing master mix and tubes with template added to the master mix at colder temperatures while carrying out downstream processing prior to nucleic acid amplification.

The process tray (900) is provided with a chiller block (902-903) attachment, which helps to maintain the solutions at desired temperatures. It is designed keeping in mind the desk space & ease processing the test. This makes it easier for the lab technician to conduct the tests within proper set ups as well as at point of care places. The chiller block (902-903) is specifically designed for solutions to be kept much below room temperature. The chiller block (902-903) is designed such that it can be directly placed in the refrigerator.

In one of the embodiments, the process tray is provided with a mapping sheet (1000) which is marked with specific colour-codes for individual steps and easy-to-understand instructions for the end user to carry out sample processing

In accordance with above embodiment, the process for detection of viral nucleic acid in a sample comprises of following steps.

1. Direct collection of biological samples from infected individuals.

Accordingly, the primary biological sample collected is saliva but other such samples such as nasopharyngeal or nasal swabs, oropharyngeal or throat swabs, buccal swabs, or any other suitable biological specimen that may be used to detect infection may also be collected.

In some embodiments, the biological sample to be collected may be collected in Viral Transport Media (VTM) or any such specific media used for the collection of biological specimens.

In other embodiments, the biological specimen may be collected without any specific media or in sterile water or buffered saline.

2. Inactivating the sample by a heat step or reagents without the need for any further procedures.

In some embodiments, the process for inactivation of biological specimens may be done heating the sample at temperature ranging from 55-95 degrees Celsius for 2-60 minutes.

In some other embodiments, the inactivation of biological samples may also be carried out using detergents, denaturants, or chaotropic agents known to lyse virions or bacterial/fungal cells, including, but not limited to, Triton X-100, Tween-20, Guanidine thiocyanate, Trizol etc.

In some other embodiments, the inactivation of the sample may also be carried out using combination of specific detergents, denaturants, or chaotropic agents and a heating at temperature ranging from 55-95 degrees Celsius for 2-60 minutes.

3. Sample processing using a specialized Process Tray that enables the end user to follow 10-12 simplified steps and minimize chances of errors.

The sample processing tray has 10-12 steps for the end user to follow with a downward flow of tubes once each step is completed. The tray has specific instructions to follow, such that the process from sample inactivation to detection of nucleic acid amplification is carried out with minimum errors.

4. Isolating the viral nucleic acid, which is then enhanced by using an enhancing reagent, which may contain non-human carrier nucleic acids that help improve the sensitivity of the detection methodology.

The viral nucleic acid isolation is enhanced using an enhancing reagent, wherein a volume between 5-50 μL of the inactivated specimen is added to an equivalent volume of the enhancing reagent and mixed by either trituration, vortex followed by a short spin or tapping manually or a combination of these. The solution is then incubated at temperatures ranging from 37-95 degrees Celsius for 5-20 minutes. In some embodiments, the incubation temperatures may follow a step-wise increase in temperature starting with 37 degrees Celsius, followed by 60 degrees Celsius followed by 95 degrees Celsius. In some embodiments, the incubation may happen using multiple combinations of temperatures and time. In other embodiments, the incubation may be only at a single temperature of 95 degrees Celsius.

After incubation, a volume ranging from 5-10 μL of the solution from first enhancing reagent tube is transferred to the second enhancing reagent tube and mixed by either trituration, vortex followed by a short spin or tapping manually or a combination of these. The solution in the second reagent enhancing tubes may be considered as template to be used for amplification of the viral genetic material.

Once the template is added to the master mix solution, a base-line (pre-amplification) reading using the specialized detection device is taken.

5. Amplification of the viral nucleic acid is carried out by incorporating the necessary buffers, enzymes, salts etc. that are needed for amplification of nucleic acids. The solution also contains one or more sets of primers and probes required for the amplification of nucleic acids, and are specific to the subject viral genome.

A volume ranging from 2-10 μL of the solution from the second enhancing reagent tube is then transferred to a master mix containing the necessary buffers, enzymes, salts, primers and probes needed for amplification of specific viral genetic material. In some embodiments, the amplification takes place using a simple heating step at a single temperature ranging from 55-80 degrees Celsius for 30-60 minutes. In some other embodiments, the amplification may be done by heating using a thermal cycler with cycling conditions specific to the primers and probes being used for amplification of the viral genetic material for short period of time.

6. Detection of the amplified nucleic acid by using specialized portable and low-cost equipment that has potential to be used as a POC device.

After the heating/thermal cycling conditions are completed, the detection device is used to determine the weather amplification of viral nucleic acid has taken place using an end-point detection of fluorescence. Samples with detectable fluorescence complying with the detection device's pre-set cut off values are considered to be positive for viral infection.

FIG. 8 illustrates a flow chart of a method of detecting the presence of a target polynucleotide sequence within a biological sample, according to an embodiment. Although the method is described as being performed on the detection device SalivaSolve®, the method involves other supplementary modules that facilitate the process to detect the target polynucleotide sequence. These modules include but not limited to the sample processing tray, heating/amplification module and mixing module. The method includes receiving, at a photo detector assembly (e.g., the photo detectors), a first light signal for a first time period before the biological sample and the reagent are reacted (pre-reaction stage). The reagent is formulated to facilitate and enhance production of a colorimetric/fluorescence/light signal (which functions as an assay signal) within or from the detection module. The assay signal indicates the presence of the target polynucleotide sequence using true amplification checks through light intensities using the readings of light intensity received from sensor for the pre-reaction and post-reaction while normalising for sample dependent background light intensities. In some embodiments, the difference in the value of light intensities indicate that the target polynucleotide sequence is present when a difference between the first value of light intensity and the second value of light intensity is within a pre-determined value range or beyond a certain pre-determined range defined by true amplification checks. For example, in some embodiments, if difference between the average value of intensity of the first light signal (i.e., the first value LI1) and the average value of intensity of the second light signal (i.e., the second value LI2) is greater than a minimum value defined by true amplification checks, then the target polynucleotide sequence is considered to be present. In some embodiments, if difference between the average value of the intensity of the first light signal (i.e., the first value of light intensity) and the average value of intensity of the second light signal (i.e., the second value of light intensity) is greater than a minimum value but less than a maximum value as defined by true amplification checks, then the target polynucleotide sequence is considered to be present. In some embodiments, the evaluation indicates that the target polynucleotide sequence is present when a ratio of the first value of light intensity and the second value of light intensity is within a predetermined ratio range defined by true amplification checks. For example, in some embodiments, if ratio between the average value of intensity of the first light signal (i.e., the first value of light intensity LI1) and the average value of intensity of the second light signal (i.e., the second value of light intensity LI2) is greater than a minimum value (e.g., fifty percent), then the target polynucleotide sequence is considered to be present.

In some embodiments, the device, the apparatus and the method are suitable for use within a point-of-care setting (e.g., doctor's office, pharmacy, hospitals, general diagnostic lab or the like). In some embodiments, the devices, the apparatus and the methods are suitable for use as an over the counter (OTC) diagnostic solution. Similarly stated, in some embodiments, the methods and devices are suitable for use by an untrained user (i.e., a lay user), can be supplied without a prescription, and can be performed independent of a health care facility (e.g., at the user's home). One of the major advantages of the said device and apparatus is that well qualified medical personnel are not necessarily required for detection of viral nucleic acids. A health worker, who is not well qualified and having less experience, will be able to carry out the tests with enough training. As demonstrated earlier, the device and the apparatus has very high mobility. Due to these factors, the device and the apparatus can also be extremely useful in remote areas, without the need of high-grade equipment.

In the current COVID-19 crisis, the said device and apparatus can play an important role to match the demands of delivering quick and accurate results. In previous peaks, existing diagnostic labs were under extreme stress, being in limited numbers. The extreme load could induce errors in the end results. The present device and apparatus are not just low-cost, it yields results that are comparable to the accuracy/sensitivity of the RT-PCR. With the present invention, the COVID-19 detection process is wrapped in around 2 hours. Further, the catch rate of the present invention is comparable to the RT-PCR. Therefore, the device and the apparatus disclosed above will be more than useful in case of exponential spread of the disease.

We claim:

1. A portable molecular diagnostic test device (1 or 2) for detection of a viral nucleic acid from an input sample comprising:
a. a housing (90) designed to contain the modules of the device;
b. a detection module (10) configured to detect the amplicon in the sample received from sensor module for true amplification checks for the presence of target amplicon with the input sample;
c. a sensor module (20) configured to receive one or more light signals and produce a sensor signal associated with the light signal;
d. a control module (30) configured for storing and executing the modules of the device (1 or 2);
e. a power module (40) designed to power the modules of the device (1 or 2);
f. a communication module (50) configured to receive an output from the controller module and forward it to an external device or a central server;
g. a display module (60) configured to take input as well provide output to the lab technician, giving a visual cue about the progression of process;
h. a feedback module (70) configured for storing & publishing the data on the device for user reference. It displays the run, sample & result of the test.

2. The device as claimed in claim 1, wherein the detection module (10) is configured for:
a. receiving, from the photodetector and optic/light sensor assembly, the value of light intensity of a first light signal for a first time period before the biological sample and reagents/enzymes are reacted (pre-heated) within the certain level of biological sample volume;
b. determining and recording in the feedback module the first value of light intensity associated with the first light signal during the first time period;
c. receiving, from the photodetector and light/optic sensor assembly, a second light signal for a second time period after the biological sample and the reagent/enzyme are reacted within a certain level of detection volume in the detection module, the second light signal associated with the assay signal;
d. determining and recording a second value of light intensity associated with the second light signal during the second time period; and
e. determining through true amplification checks and displaying through the display module/screen, based on amplifications through light intensities changes while normalising for sample dependent background light intensities, whether the target polynucleotide sequence is present in the biological sample.

3. The device as claimed in claim 1, wherein the device further comprises a feedback module (70) is configured for storing, publishing and displaying the data on the device for user reference.

4. The device as claimed in claim 1, wherein, the display module (60) comprises of a digital screen, a light output, an audible output, a wireless signal, or a haptic output or a combination thereof.

5. The device as claimed in claim 1, wherein communication module (50) is configured for transmitting data by a short-range wireless communication protocol including but not limited to Bluetooth, wireless adapter, infrared waves.

6. The device as claimed in claim 1, wherein the modules (10, 20, 30, 40, 50 and 60, 70) are standalone modules.

7. The device as claimed in claim 1, wherein the modules are integrated in a single housing (90).

8. The device as claimed in claim 1, wherein the detection module (10); the sensor module (20); the control module (30) and the communication module (50) are integrated into an electronic system.

9. The device as claimed in claim 8, wherein the electronic system further comprises a light assembly, a photo detector assembly, a memory, a processing and controlling device implemented in at least one of the memory or the processing device, the optic/light sensor positioned on one side of the detection module, the optic/light sensor configured to capture the average value of light intensity produced during the detection of the target polynucleotide sequence.

10. The device as claimed in claim 9, wherein the electronic system is configured to generate any one or more than one combination of outputs, selected from, a display output through a digital screen, a light output, an audible output, a wireless signal, or a haptic output or combination of any output mentioned herein is based on the indication whether target polynucleotide sequence is present in the detection sample volume.

11. The device as claimed in claim 8, wherein the electronic system further comprises of a photodetector assembly positioned to capture or receive a light signal, the light signal associated with any of a reflection or a luminance of the biological sample.

12. The device as claimed in claim 1, wherein, the assay signal is a colorimetric signal, a chemiluminescence signal, or a fluorescence signal.

13. The device as claimed in claim 1, wherein the detection module (10) includes a detection filter(s), and optical/light sensor which detects the assay signal being produced at the detection filter(s).

14. The device as claimed in claim 1, wherein the detection module (10) is configured to read and record the average light intensity produce by a first light during the first time and a second light during the second time, wherein the first value of light intensity associated with an average intensity of the first light, the second value of light intensity associated with average intensity of the second light signal.

15. A method for detecting a viral nucleic acid using the device as claimed in claim 1, wherein the method comprises:
a. collecting samples from individuals suspected of being infected;
b. inactivating the sample wherein the pathogen may be inactivated by heating or by using detergents, denaturants, or chaotropic agents or combinations thereof;
c. isolating the viral nucleic acid from the sample of step (a);
d. amplifying the viral nucleic acid of step (c); and
e. detecting the amplified nucleic acid of step (d),
wherein the detection of viral nucleic acid in step (e) comprises steps of:
(i) receiving and recording a first light signal for the first time period before the biological sample and the reagent/enzymes are reacted within certain levels of sample volume in the detection module (10), the first light signal associated with the first assay signal;
(ii) receiving and recording a second light signal for the second time period after the biological sample and the reagent/enzymes are reacted and amplified at isothermal or thermal cycling conditions within certain levels of sample volume in the detection module (10), the second light signal associated with the second assay signal;
(iii) determining the average value of light intensity associated with the first light signal and the second light signal during the first and second time period; and
(iv) determining and communicating, based on true amplification checks through light intensity changes using the first value of light intensity and the second value of light intensity while normalising for sample dependent background light intensities, if the target polynucleotide sequence is present in the biological sample, and
(v) producing an electronic output when the true amplification checks using the first value of light intensity and the second value of light intensity indicates that the target polynucleotide sequence is present.

16. The method as claimed in claim 15, wherein the amplification of the viral nucleic acid is carried out by isothermal heating or thermal cycling at temperatures ranging from 55-90 degrees Celsius for 30-60 minutes.

17. The method as claimed in claim 15, wherein the step (c) further comprises of incubating the sample at temperatures ranging from 37-95 degrees Celsius for 5-20 minutes for inactivation of the pathogen.

18. The method as claimed in claim 15, wherein the step (c) further comprises of incubating the sample at a step-wise increasing temperature, starting with 37 degrees Celsius, followed by 60 degrees Celsius followed by 95 degrees Celsius for inactivation of the pathogen.

19. The method as claimed in claim 15, wherein the sample is any tissue or fluid obtained from a pathogen containing nucleic acid.

20. An apparatus for detection of the viral nucleic from an input biological sample comprising:
a. a sample tube (104) configured to receive the input biological sample;
b. at least one process tray (900) configured for processing the input biological sample and emitting plurality of assay light signals indicating the presence of a viral nucleic acid, wherein the process tray is configured (i) to store at least signal enhancing reagent, (ii) to allow efficient mixing of the input biological sample and various solutions, (iii) to display a user specific instruction, and
c. the portable device (1 or 2) for detection of viral nucleic acid as claimed in claim 1; wherein the detection of the viral nucleic acid takes place by a method comprising:
(i) receiving, from the photodetector and optic/light sensor assembly, the value of light intensity of a first light signal for a first time period before the biological sample and reagents/enzymes are reacted (pre-heated) within the certain level of biological sample volume;
(ii) determining and recording in the feedback module the first value of light intensity associated with the first light signal during the first time period;
(iii) receiving, from the photodetector and light/optic sensor assembly, a second light signal for a second time period after the biological sample and the reagent/enzyme are reacted and amplified at isothermal or thermal cycling conditions within a certain level of detection volume in the detection module, the second light signal associated with the assay signal;
(iv) determining and recording a second value of light intensity associated with the second light signal during the second time period; and
(v) determining through true amplification checks and displaying through the display module/screen, based on amplifications through light intensities changes while normalising for sample dependent background light intensities, whether the target polynucleotide sequence is present in the biological sample.

21. The apparatus as claimed in claim 20, wherein the process tray comprises of (a) plurality of insertion holes (901) configured for receiving tubes (104) containing the reagents, enzymes, master mix and target sequences; (b) plurality of control tubes, and (c) process mapping sheet (1000).

22. The apparatus as claimed in claim 21, wherein the reagent comprises two lysis-based solutions which are RNA/DNA enhancing reagents and enzymes.

23. The device as claimed in claim 21, the master mix comprises a reference polynucleotide sequence along with the probe and quencher which is at least one of a control polynucleotide sequence or an invariant polynucleotide sequence associated with the target polynucleotide sequence that is used before the first assay signal to record the baseline.

24. The system as claimed in claim 21, wherein the at least one process tray (900) is optionally provided with a cooling unit (902-903) to maintain various solutions at a temperature lower than a room temperature.
